(19) United States
(12) Patent Application Publication
(10) Pub. No.: US 2026/0272828 A1
(43) Pub. Date: Sep. 17, 2026

(54) Sterile formulation of an anesthetic and bronchodilator

(71) Applicant: Steven Warrington, North Sioux City, SD (US)

(72) Inventor: Steven Warrington, North Sioux City, SD (US)

(21) Appl. No.: 19/076,948

(22) Filed: Mar. 11, 2025

(51) Int. Cl. A61K 9/00; A61K 31/137; A61K 31/167; A61K 31/56

(52) U.S. Cl. CPC A61K 9/0078; A61K 31/137; A61K 31/167; A61K 31/56

(57) ABSTRACT

This invention is a sterile formulation of an anesthetic and bronchodilator. It can be used through multiple methods. Some embodiments contain an additional therapeutic agent with the ultimate formulation remaining sterile. Some embodiments are diluted in a sterile solution with the ultimate diluted formulation remaining sterile. Some embodiments are specifically intended for use by delivery to the respiratory, oropharyngeal, and/or nasopharyngeal tract.

### CROSS-REFERENCE TO RELATED APPLICATION

None/Not applicable

### STATEMENT OF FEDERALLY SPONSORED RESEARCH OR DEVELOPMENT

This was not federally sponsored research. This was not federally developed.

### NAMES OF PARTIES TO JOINT RESEARCH AGREEMENT

None/Not Applicable.

SEQUENCE LISTING

None/Not applicable

### BACKGROUND OF THE INVENTION

### Field of invention

The present invention provides a novel formulation to be used for unwanted body processes such as, but not limited to, asthma, pain, sore throat, cough, and bronchoconstriction. This may be used such as, but not limited to, prevention, alleviation, therapy, and/or treatment. Such formulation can be used multiple ways such as, but not limited to, nebulization, atomization, intranasal, metered dose inhaler, dry powder inhaler, and vaporization.

Many individuals deal with unwanted body processes. These can be from many reasons such as, but not limited to, injury, infection, due to a procedure, due to an underlying disease, as a result of another medical condition, and occurring without a known cause.

There are multiple treatments available depending on the unwanted process present with one area of treatments being therapeutic substances. These can include anesthetics, bronchodilators, corticosteroids, and many others.

Anesthetics are one such group of medications that may be beneficial. For example, lidocaine has been shown to have multiple potential uses such as, but not limited to, pain, bronchospasm, cough, and use in asthma (Groeben et al., 2000; Lv et al., 2011; Partovinezhad, et al. 2024; Welter et al., 2023). Slaton et al. (2013) provides a summary and highlights numerous articles that have looked at nebulized lidocaine specifically, as well as commenting on those that compare it to bronchodilators such as by Chong et al. (2005). Of note, the only one that uses combination therapy in that is discussed by Groeben et al. (2000), which is discussed elsewhere in the application.

Certain medications are unique in being anesthetics with potential bronchodilator capabilities. Ketamine is an anesthetic medication and works through N-methyl-D-aspartate receptor antagonist (Ekong et al., 2025). Ketamine is unique though in that it also “shows potential as a bronchodilator” (Binsaeedu et al., 2023). Dexmedetomidine is another unique medication that is an anesthetic. There is debate in literature on the bronchodilatory nature as Motamed et al. (2021) note it “had no effect on the improvement of spirometry parameters and RR of patients” translating to not having bronchodilatory properties, while Bohringer and Liu (2019) note it does have a bronchodilatory effect.

Bronchodilators are another group of medications with multiple groups or classes of medications that can be considered bronchodilators. Examples of bronchodilators include those such as, but not limited to, short acting beta-2 agonists, anticholinergics, leukotriene receptor antagonists, and magnesium. Bronchodilators have multiple potential uses such as, but not limited to, asthma, chronic obstructive pulmonary disease, and cough (Lowry et al., 1994; Sriprasart et al., 2023; van Geffen et al., 2017).

There are numerous additional medications that can be inhaled to prevent or address unwanted effects such as, but not limited to, corticosteroids, statins, and sympathomimetics.

Sometimes multiple therapies are required, such as the use of a short acting beta agonist followed by the use of a local anesthetic. Hoffman et al. published in 2006 using albuterol as a pre-treatment to lidocaine inhalation, but not as combination therapy. Such process of pre-treatment using a short acting beta agonist prior to using a local anesthetic had been previously discussed to an extent similarly in the research article by Harrison and Tattersfield (1998).

There are also treatments that combine therapies, such as bronchodilators and steroids being combined and delivered at the same time. One combination treatment that has been described from a few research papers is the use of bronchodilator and anesthetic. One such example is the use of salbutamol, a short acting beta agonist bronchodilator, in combination with lidocaine, an anesthetic. Groeben et al. (2000) demonstrated the benefit of salbutamol and lidocaine as a combination treatment through a lengthy process requiring significant additional equipment and materials. Unfortunately, it had multiple problems that the current invention will address. First, their process was not user friendly or easy to be done away from additional medical devices and/or a hospital as they required delivery by using compressed air nebulization at 30 lb/sq inch. Second, in their process they required dilution with an additive free saline solution, and required initiation using triggering by a flowmeter. Third, it required preparation at time of therapy. Fourth, it did not use a sterile saline, which is further discussed later in the application.

Thomson (1979) discusses anesthetic with bronchodilator therapy, however has problems the current invention addresses. For instance, the medications were administered sequentially rather than combined, had varying concentrations, and used compressed air.

Similar to Groeben et al. (2000), Partovinezhad et al. (2024) published a research article on a therapy that included a substance which could be considered a bronchodilator, ketamine, in combination with the anesthetic lidocaine. Unfortunately, it too had multiple problems that the current invention will address. Again, their process was not user friendly or easy. They used weight based individualized dosing through preparation at time of use. Specifically, for nebulization they had separate syringes of ketamine and lidocaine that were then added together at time of use. There are also issues surrounding a lack of sterility which is discussed in this application as well. Each of the substances were in different concentrations, and used weight-based dosing. That is an additional problem to be addressed by the current invention, as it is an additional step in a process leading to increased potential for errors, which could cause harm to the individual using the therapy.

Another published article on a therapy that includes a substance that as mentioned above could be considered a bronchodilator, ketamine, in combination with a substance that could be considered an anesthetic, clonidine, was done by Shekhar et al. (2019). A first issue is that the process used a compressor nebulizer, making it potentially difficult to do depending on the location and equipment available. A second issue is that the formulation was prepared at time of use, which can again prolong time associated with therapy and delay time to therapy. Finally, it did not use a sterile saline, which is a problem that the current invention addresses and is further discussed later in this application. A similar substance was documented by Jain et al. (2017) with multiple problems that the current invention addresses. Problems included not using sterile saline and preparation at time of therapy.

There has also been documentation of a formulation that could be considered an anesthetic, dexmedetomidine, and a potential bronchodilator, ketamine. These documentations also have problems which are addressed by the current invention. For instance, Zanaty et al. (2015) had problems including not using sterile saline, preparation near time of administration, weight-based dosing of each substance at different amounts (e.g., 1 microgram/kilogram of one and 1 milligram/kilogram of the other), which creates potential for error and harm, and required the use of a “standard hospital jet nebulizer.” Another of these documentations is by Sabry et al. (2020), which had problems including using a “wall nebulizer” at a hospital, not using sterile saline, and requiring preparation with weight-based dosing at different amounts (e.g., 1.5 microgram/kilogram of one and 1.5 milligram/kilogram of the other). Another documentation by Dharamkhele et al. (2022) had problems including using weight-based dosing at different amounts (e.g., 1 microgram/kilogram of one and 1 milligram/kilogram of the other), not using sterile saline, use of a “hospital jet nebuliser.” Another documentation by Sabra (2018) delivered the medications intravenously, issues surrounding a lack of sterility discussed elsewhere in this application, and required additional care surrounding administration, a problem that the current invention solves.

Another potential anesthetic and bronchodilator combination therapy has been documented by Gu et al. (2019) of lidocaine as an anesthetic and dexmedetomidine if it is considered to be a bronchodilator. Their documentation however runs into problems solved by the current invention such as surrounding preparation for administration at time of therapy that can delay therapy, issues surrounding a lack of sterility discussed elsewhere in this application, and preparation of the substances by users creating an extra step and potential for error and harm.

Another combination therapy has been documented in the systemic review by Lin et al. (2022) surrounding dexmedetomidine including potential anesthetic and potential bronchodilator combinations. They performed a systemic search, with their individual results analyzed and each demonstrating problems the current invention solves. For instance, Sado-Filho et al. (2021) required preparation of a weight-based dose using varying concentrations (e.g., 2 micrograms/kilogram of one and 1 milligram/kilogram of another), along with using injectable solutions that would require mixing for combination leading to potential errors, along with actually delivering the separate components 20 minutes apart and prolonging treatment time rather than as truly combined therapy, along with issues surrounding a lack of sterility. Praveen et al. (2023) documented use of dexmedetomidine with lidocaine, however have problems associated with preparation at time of use and varying concentrations of each (1.5 microgram/kilogram of one and varying amount of another). Again, there are also issues surrounding a lack of sterility. Zanaty et al. (2015) is also mentioned by Lin et al., but is discussed elsewhere in this application. Again, there are also issues surrounding a lack of sterility.

There are also combination therapies when one medication is delivered topically to the intended area of the respiratory and/or nasopharyngeal and/or oropharyngeal tract (e.g., nebulization, gargle, atomization) with the other medication given by an intravenous route, such as by Fang et al. (2019) or Safavi et al. (2014). These come with the problems surrounding administration and requiring an IV for the individual getting therapy and are not actually mixed or combined. Ekong et al.'s review also carries the issue of having no formulations that allowed strictly inhalational therapy.

There are also combinations when one medication is delivered topically via one route (e.g., nebulization) and another mediation is administered another non-intravenous route (e.g., topical direct). An example is the bronchodilator nebulized and anesthetic topical discussed by Wang et al. (2019). Again, there are problems with that, which the current invention solves surrounding administration and potential for errors due to additional steps.

There are also limited bench research studies that have also evaluated anesthetic and bronchodilator combinations. One such example is seen in the article by Yunoki et al. (2003) in which they discuss competition of a substance that could be considered a bronchodilator to cells in the presence and absence of an anesthetic for radioligand receptor binding assay of hamster ovary cells. There are multiple problems the current invention solves from bench research substances, such as surrounding administration and concentration/amount differences, sterility, and preparation prior to the use.

To understand the problems solved, it is important to understand problems associated with medications, as they can cause potentially avoidable harm to an individual (Tetteh, 2019).

One problem is associated with deviation from an intended treatment protocol (Tetteh, 2019). One specific example is if an individual receives to little or too much medication(s).

Reducing the number of steps in a process, such as not requiring mixing multiple medications at time of therapy, can reduce the potential for errors, and in turn reduce potential for harm (Six Sigma, 2024).

Another potential deviation from the intended protocol is with an individual's ability to adhere to it. There are many reasons why an individual may not adhere to a protocol. One such reason is complexity. For example, one study demonstrated approximately 10% of individuals had issues with administration and use of nebulization of a substance (Lim et al., 2013). Complexity and number of drugs involved are two of many reasons that may decrease adherence to a protocol intentionally or unintentionally (Lee et al., 2018).

Of particular importance to the discussion of novelty of the current invention is that not all pharmaceuticals are sterile. This includes sterility of saline and sterility of pharmaceuticals and/or medications and/or substances. The use of nonsterile saline and/or pharmaceuticals and/or products have potential for contamination and harm (Tetteh, 2019). There are times when a nonsterile saline and/or pharmaceutical is acceptable, such as a topical solution to a pressure ulcer as noted by Xakellis and Chrischilles (1992). However, the use of nonsterile solution can also cause significant harm and death (Schroeder et al., 2015). Nonsterile saline has been documented by more than one author, and the use of it has been documented to cause harm and death depending on the use (Jenkins & Phillips, 1986; Schroeder et al., 2015). This invention solves this issue as it is a sterile formulation, such as even after having used saline solution such as for dilution. Also of importance, Mugoyela and Mwambete (2010) found multiple nonsterile pharmaceuticals and/or medications that are in use. This invention solves this issue as it is a sterile formulation. Nonsterile issues can also lead to delivery of a bacterial or pathogenic aerosol that can cause harm (Craven et al., 1984; Harris et al., 2002; Vassal et al., 2000).

It is important to note that one can't assume a saline solution, or a normal saline solution, is sterile, as that has led to multiple deaths (Schroeder et al., 2015). Again, this invention solves this issue as it is a sterile formulation.

It is important to note that there have been better outcomes shown with delivery of combinations of medications at once rather than via separate administration (Price et al., 2016).

Another problem is the time taken to treat a patient as a longer time to beginning treatment has been shown to be associated with worse outcomes for an individual (Saver et al., 2013; Selroos et al., 1995). This invention is an improvement as it is already combined and ready when an individual needs it.

To address another area that the current invention improves upon is what may be called an anesthesia gas machine (also known by other naumes such as an anesthesia machine, anesthesia workstation, anesthesia delivery station, anesthesia delivery system, Boyle's machine, and Boyle's apparatus). This machine may at times allow combining substances that could be considered anesthetics and bronchodilators, however sterility is not a certainty, and substances are combined at time of use.

Multiple problems are solved by the current invention.

One problem is that certain substances used for therapy, such as the anesthetic lidocaine, can also produce unwanted effects, requiring additional medications, such as the pretreatment by a bronchodilator, such as salbutamol. Such has been discussed by Harrison and Tattersfield (1998). The current invention addresses that problem by combining anesthetic and bronchodilator to address unwanted effects and risks such as bronchoconstriction.

A second problem surrounds requiring preparation at time of use and the issue of intentional and/or unintentional deviation from the protocol. There is risk of deviation from the protocol with both too much and/or too little if incorrectly prepared, which can each create harm. This invention may prevent certain deviations. For instance, having more steps in a process, such as an individual having to prepare at time of use, is associated with more chances of error. Additionally, it prevents individuals from choosing to only use one medication when the intended protocol is more than one medication. Additionally, it may prevent unintentional errors in preparation of protocol ratio. Additionally, it may also prevent deviation from protocol due to nonadherence secondary to perceived complexity of preparation. This invention addresses the problem by providing already prepared combined anesthetic and bronchodilator ready for use.

A third problem surrounds requiring preparation at time of use and the issue of delaying initiation of treatment, and prolonging time to treatment completion. This invention addresses the problem by providing already prepared anesthetic and bronchodilator ready for use.

A fourth problem is when dilution is required and contamination and/or unwanted effects and/or unintended effects occur due to nonsterile products being used, such as using nonsterile saline versus sterile saline. As discussed elsewhere in this patent application, the use of nonsterile saline solution, and the assumption that saline solution is sterile, has led to multiple deaths (Schroeder et al., 2015). This invention addresses that problem as it is a sterile formulation.

A fifth problem surrounds adherence when using multiple medications. It has been noted by Dhand et al. (2018) that it may be preferable to deliver them all rather than each singularly and potentially through different devices. Combining medications may increase compliance, and could decrease some issues associated with the device(s) or method(s) used for delivery of medication(s). This invention addresses that issue by delivering multiple intended medications simultaneously.

A sixth problem is that the method of delivery can be impactful from both the patient and physician's perspective as documented by Dhand et al. (2018). There are numerous potential methods for delivery such as, but not limited to, nebulization, vaporization, metered dose inhalers, etc . . . This invention addresses that problem as it allows some choice to the method of delivery.

It should be noted in reviewing the field of invention, and this application, that the use of numbering in the format of [XXXX] is provided to help a reader of the patent. In the event that there is more than one of a single number (e.g., [0001] and a second paragraph labeled [0001]), those sections may be read together and/or combined. It should also be noted in the event of no numbered paragraph where one may be expected (e.g., [0001] and [0003] but no [0002] is found), it can be used as a break or additional space between known paragraphs. It should also be noted that times a statement may be without a numbered paragraph, and in those cases it could be read as an independent line such as a header for a following section (e.g., “BACKGROUND OF THE INVENTION”), or a separate section in which alternative numbering or expectations are used such as in the claims section where no [XXXX] is to be expected in this application. It should also be noted that at times a statement may be without a period following it, and in those cases it could be read as an independent line such as a header for a following section (e.g., “BACKGROUND OF THE INVENTION”), or as a list item (INVENTOR: Steven Warrington in which Steven Warrington is the only list item following the INVENTOR:).

### BRIEF SUMMARY OF THE INVENTION

This invention is a sterile formulation of an anesthetic and bronchodilator. It may also include an additional therapeutic agent. It may be intended for use by delivery to the respiratory, oropharyngeal, and/or nasopharyngeal tract. It includes at least one anesthetic and at least one bronchodilator, each being an effective dose. Certain embodiments may also include additional therapeutic agents. Certain embodiments are diluted with a solution. The sterile formulation may be produced to be delivered in multiple methods such as, but not limited to, nebulization, vaporization, atomization, dry powder inhalation, and metered dose inhalation. The formulation may be produced in a single dose container or a multi-dose container. Advantages of the invention include: a) it is combined prior to reaching intended consumer(s), which may prevent and/or decrease errors; b) it is combined prior to reaching intended consumer(s), which may prevent and/or decrease adherence issues; c) it is combined prior to reaching intended consumer(s), which may improve outcomes; d) it is combined prior to reaching intended consumer(s), which may prevent and/or decrease deviation in therapy; e) it is combined and ready for use, which may decrease time taken to initiation of therapy once a decision for use is made; f) it is combined and ready for use, which may decrease time taken to completion of therapy once a decision for use is made; g) it is sterile, which may prevent and/or decrease harm to the individual(s) using it; h) it is able to be delivered by various methods, which may increase adherence of use; i) it is able to be delivered by various methods, which may increase potential settings of use.

### DETAILED DESCRIPTION OF THE INVENTION

The terminology used herein is for the purpose of describing particular embodiments only and is not intended to be limiting of the invention. Described embodiments are not the only embodiments. The term “such as” is used to provide an example, or multiple examples, but does not limit to that example/those examples provided. As used herein, the term “and/or” includes any and all combinations of one or more of the associated listed items. It will be further understood that the term “e.g.” refers to “for example,” and provides an example, but does not limit to only that example. It will be further understood that the terms “medication,” “medicine,” “pharmaceutical,” “formulation,” “drug,” and “compound” may be used somewhat interchangeably. It will be further understood that the terms “may,” and “can” may be used somewhat interchangeably. It will be further understood that the terms such as “mix,” “blend,” and “combine” may be used somewhat interchangeably.

It will be further understood that the ratio of anesthetic to bronchodilator may change depending on the substance(s) used to be an effective dose and those discussed herein are but one preferred embodiment. It will also be understood that there may be multiple effective doses of each anesthetic and/or bronchodilator and/or additional therapeutic compound depending on the effect(s) desired. It will also be understood that substances may be combined for a single dose or in a larger amount to permit multiple doses to be combined at once. It will also be understood that substances may be packaged in multiple formats for various delivery methods and may be packaged in multiple formats to allow for single or multiple doses in a single package. It will also be understood that there are multiple devices and methods that may be able to mix substances, and that use of those devices and descriptions of mixing such as amounts of substance(s) included for mixing and/or time chosen to mix substances may vary dependent on the mixing device used and mixture characteristics desired, but different embodiments may use alternative processes and/or methods and/or devices and/or techniques to mix.

The present disclosures are to be considered as an exemplification and examples of the invention, and are not intended to limit the invention to the specific embodiments and/or descriptions below.

Anesthetics are those such as, but not limited to, benzocaine, bupivacaine, clonidine, etomidate, isoflurane, ketamine, lidocaine, prilocaine, proparacaine, propofol, ropivacaine, tetracaine.

Bronchodilators are those such as, but not limited to, formoterol, levalbuterol, montelukast, ipratropium, ketamine, magnesium salbuterol, salmeterol, terbutaline, theophylline, and tiotroprium. Of note, magnesium can be a bronchodilator and has “no primary analgesic activity” (James, 2009; Knightly et al., 2017). Knightly et al. (2017) provide a lengthy discussion on some of the benefits of magnesium. Ekong et al. (2025) also discusses some of magnesium's benefits.

Additional therapeutic substances include those from classes such as, but are not limited to alpha1-Proteinase Inhibitor, antibiotic, antiviral, corticosteroid, mucolytic, statin, surfactant.

Production of anesthetics, bronchodilators, and other therapeutic substances are well-documented.

This invention's specific area of improvement surrounds sterility. There are numerous bronchodilators. There are numerous anesthetics. There are numerous therapeutic agents that may have potential benefit as additives. There are numerous known methods to produce bronchodilators, anesthetics, and potential added therapeutic agents. Consider for example the compound ketamine, which as discussed earlier could be considered to be an anesthetic, could also be considered to have bronchodilatory properties and considered as a bronchodilator for the purposes herein, or could be used as an additional therapeutic agent to be added to an anesthetic and a bronchodilator. Examples of synthetic routes of ketamine include those such as, but not limited to, those described by patent U.S. Pat. Nos. 3,254,124A, 11,440,874B2, Zekri et al. (2020), Parcell and Sanchez (1981), Elhawi et al. (2012), Yen et al. (2023), Trauner and Ko (2023), Weber et al. (2023), which are incorporated herein by reference in their entirety. Further, there are more representative examples that are also known and available to those skilled in the art when considering the potential variants of ketamine such as, but not limited to, enantiomers, prodrugs, or intermediaries with some representative examples of those synthetic routes including patent U.S. Pat. Nos. 10,815,196B2 and 11,723,883B2. Each substance (e.g., anesthetic, bronchodilator, other therapeutic additive) and their known potential variants such as, but not limited to salts, enantiomers, and intermediaries, have representative examples known and available to those skilled in the art. Additionally, there are also further representative modifications that are also known and available to those skilled in the art such as seen by patent RU2497507C2. Other suitable methods for synthesizing ketamine and/or ketamine variants including but not limited to prodrugs, intermediaries, and enantiomers, are known in the art and have been described.

To highlight the novelty of this patent's invention, not all pharmaceuticals including, but not limited to, saline and/or medications are sterile as discussed in this application in [0023] and [0024] . In the preceding paragraph, in all of those materials referenced related to synthesis of ketamine, only patent U.S. Pat. No. 10,815,196B2 discusses sterility. Even in that example, sterility is discussed as: a) the carrier: “For parenteral administration, the carrier will usually consist of sterile water,” “For parenterals, the carrier will usually comprise sterile water,”; and b) in relation to the parenteral route: “such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation.,” “forms useful for parenteral administration include sterile solutions, emulsions and suspensions.,” “For parenteral administration, sterile suspensions and solutions are desired.” There is no distinct note of a formulation that is ultimately sterile. Despite those being the result of multiple searches, another search was undertaken and identified a website who noted “Lab-scale synthesis is primarily responsible for producing ketamine. Highly controlled environments, such as research and pharmaceutical laboratories, develop the substance following strict protocols and safety measures. These specialized facilities ensure the purity and potency of the final product. Within these laboratories, scientists and chemists meticulously carry out the production process. They work in sterile conditions, wearing protective gear to prevent any contamination. The laboratories are equipped with state-of-the-art equipment and instruments, allowing precise measurements and control over the chemical reactions” (Southern Ketamine, 2023). In that material, they note a sterile work condition, but do not identify the actual ketamine as sterile, or production of a sterile formulation. In another search, Wa and Ping (ND) found a paper on ketamine that comments on one diluent being sterile “Pharmaceutically acceptable diluents include sterile water, saline, buffered saline, dextrose solution.” Again, it is not of a sterile formulation that is ultimately produced.

It is noted that there are multiple representative examples and methods that are also known and available to those skilled in the art related to sterility such as, but not limited to, producing a sterile product, and/or sterilizing a product, and/or packaging a sterile product. Examples include, but are not limited to, patent WO2017158473A1, patent ES2960921T3,patent WO2017158473A1, patent RU2700162C2, patent US20190269764A1, patent JP6290625B2, patent CA2374257A1, Kumar and Rana (2021), Yaman (2018), Searles and Ohtake (2021), Phogat (2024), which are incorporated herein by reference in their entirety.

It is also noted that there are multiple representative examples and methods that are also known and available to those skilled in the art related to sterilization such as, but not limited to, using thermal modification (heat or cold) and/or radiation, which are incorporated herein by reference in their entirety (CDC, 2008; Yang et al. 2007).

It is noted that there are multiple representative examples and methods that are also known and available to those skilled in the art related to mixing two or more substances. Examples include those such as, but not limited to, Spahn et al. (2022), Brone et al. (1997), and AIPAK pharma (ND), which are incorporated herein by reference in their entirety.

It is also noted that there are multiple representative examples and methods that are also known and available to those skilled in the art related to carriers and/or excipients. Examples include those such as, but not limited to, Saharan et al. (2008), patent CA2909357C, and patent US20190365672A1, which are incorporated herein by reference in their entirety.

It is important to note that certain embodiments may use components of the formulation that can be purchased such as purchasing a component lidocaine as a dry powder formulation and the component salbutamol as the dry powder formulation rather than synthesizing each of those components. It is important to understand that such purchased component may already be sterile. However, in that situation they would require combination and/or mixing into a product with a formulation that is ultimately sterile. It is also important to understand that the current invention could use purchased components such as a dry powder formulation of lidocaine that requires sterilization prior to mixing, or following mixing, to yield an ultimately sterile formulation. It is noted that there may be impurities regarding purchased substances and/or only a percentage of a compound may be the intended substance (e.g., 98% lidocaine), and that for certain embodiments it may still be purchased and used. It is also important to understand that certain embodiments could require sterilization as a final step.

In the present invention, the anesthetic and bronchodilator may be prepared, and/or mixed, and/or diluted, and/or packaged using known methods, however adapted and/or combined as necessary to result in a formulation containing at least one anesthetic and at least one bronchodilator with the ultimate formulation being sterile. In embodiments related to claims that include an additional therapeutic compound, that additional therapeutic compound and/or addition of a therapeutic compound is also adapted as necessary to result in a formulation containing at least one anesthetic and at least one bronchodilator and at least one additional therapeutic compound with the contained formulation being sterile.

Sterility can be tested by multiple methods such as, but not limited to, USP 71 (Certified Laboratories, ND), USP 797 (Taylor, ND), and/or inoculation and/or filtration methods (Charles River, ND). Sterility in regard to a formulation can refer to freedom from the presence of viable microorganisms (World Health Organization, ND).

In one preferred embodiment, the anesthetic lidocaine is purchased in a dry powder form at a concentration of at least 99% purity that is known to be sterile and the bronchodilator salbutamol is purchased in a dry powder form of at least 99% purity that is known to be sterile and is mixed using sterile precautions of limited lab/environment access, lab coat, gloves, mask, having cleaned hands prior to work, previously sterilized equipment, and a work area that was previously wiped down with 70% ethanol. Dry powders of lidocaine 40 mg and salbutamol 1.5 mg are mixed using a V dry powder mixer for 15 minutes. The mixture is then placed into a sterile glass vial and sealed with a sterile hermetic fitted lid.

In one preferred embodiment, the anesthetic lidocaine is purchased in a dry powder form at a concentration of at least 99% purity that is known to be sterile and the bronchodilator salbutamol is purchased in a dry powder form of at least 99% purity that is known to be sterile and the additional therapeutic fluticasone is purchased in a dry powder form of at least 99% purity that is known to be sterile and those three are mixed using sterile precautions of limited lab/environment access, lab coat, gloves, mask, having cleaned hands prior to work, previously sterilized equipment, and a work area that was previously wiped down with 70% ethanol. Dry powders of lidocaine 40 mg and salbutamol 1.5 mg and fluticasone 113 micrograms are mixed using a V dry powder mixer for 15 minutes. The mixture is then placed into a sterile glass vial and sealed with a sterile hermetic fitted lid.

In one preferred embodiment, the anesthetic lidocaine is purchased in a dry powder form at a concentration of at least 99% purity that is known to be sterile and the bronchodilator salbutamol is purchased in a dry powder form of at least 99% purity that is known to be sterile is mixed using sterile precautions including limited lab/environment access, lab coat, gloves, mask, having cleaned hands prior to work, previously sterilized equipment, and a work area that was previously wiped down with 70% ethanol. Dry powders of lidocaine 40 mg and salbutamol 1.5 mg are mixed using a V dry powder mixer for 15 minutes. The mixture is then transferred to a sterile glass container and is diluted with normal saline that is known to be sterile to a total volume of 5 milliliters. The mixture is then sealed with a sterile hermetic fitted lid.

In one preferred embodiment, the anesthetic lidocaine is purchased in a dry powder form at a concentration of at least 99% purity that is known to be sterile and the bronchodilator salbutamol is purchased in a dry powder form of at least 99% purity that is known to be sterile and the additional therapeutic fluticasone is purchased in a dry powder form of at least 99% purity that is known to be sterile and those three are mixed using sterile precautions including limited lab/environment access, lab coat, gloves, mask, having cleaned hands prior to work, previously sterilized equipment, and a work area that was previously wiped down with 70% ethanol. Dry powders of lidocaine 40 mg and salbutamol 1.5 mg and 113 micrograms fluticasone are mixed using a V dry powder mixer for 15 minutes. The mixture is then transferred to a sterile glass container and is diluted with normal saline that is known to be sterile to a total volume of 5 milliliters. The mixture is then sealed with a sterile hermetic fitted lid.

1. A sterile formulation that includes
an effective amount of at least one local anesthetic or a pharmaceutically acceptable salt thereof, and
an effective amount of at least one anticholinergic bronchodilator or a pharmaceutically acceptable salt thereof.

2. A sterile formulation as defined in claim 1, wherein the anticholinergic bronchodilator is ipratropium and/or tiotropium.

3. A sterile formulation as defined in claim 1 that is intended for administration topically to the respiratory, oropharyngeal, and/or nasopharyngeal tract.

4. A sterile formulation as defined in claim 1 that includes at least one additional therapeutic substance.

5. A sterile formulation as defined in claim 1 that does not require a compressed air stream for an individual to use.

6. A sterile formulation as defined in claim 1 that does not require a compressed air stream for an individual's use and includes at least one additional therapeutic substance and remains sterile when packaged in sterile packaging when the formulation is tested by USP 71 sterility testing at least 24 hours after production.

7. A sterile formulation that includes
an effective amount of at least one local anesthetic or a pharmaceutically acceptable salt thereof, and
an effective amount of at least one short acting beta-2 agonist bronchodilator or a pharmaceutically acceptable salt thereof.

8. A sterile formulation as defined in claim 7 wherein the short acting beta-2-agonist bronchodilator is levalbuterol and/or salbutamol.

9. A sterile formulation as defined in claim 7 that includes at least one additional therapeutic substance.

10. A sterile formulation as defined in claim 7 that is intended for administration topically to the respiratory, oropharyngeal, and/or nasopharyngeal tract.

11. A sterile formulation as defined in claim 7 that does not require a compressed air stream for an individual's use.

12. A sterile formulation that includes
an effective amount of at least one local anesthetic or a pharmaceutically acceptable salt thereof, and
an effective amount of at least one bronchodilator that is chosen from ipratropium, tiotropium, formoterol, levalbuterol, salbutamol, salmeterol, and/or terbutaline, or a pharmaceutically acceptable salt thereof.

13. A sterile formulation as defined in claim 12 that is intended for inhalational administration.

14. A sterile formulation as defined in claim 12 that is intended for administration topically to the respiratory, oropharyngeal, and/or nasopharyngeal tract.

15. A sterile formulation as defined in claim 12 that includes at least one additional therapeutic substance.

16. A sterile formulation as defined in claim 12 that does not require a compressed air stream for an individual to use.

17. A sterile formulation as defined in claim 12 that does not require a compressed air stream for an individual's use and remains sterile when packaged in sterile packaging when the formulation is tested by USP 71 sterility testing at least 24 hours after production.

18. A sterile formulation as defined in claim 12 that is diluted in a sterile solution, and intended for administration topically to the respiratory, oropharyngeal, and/or nasopharyngeal tract and and remains sterile when packaged in sterile packaging when the formulation is tested by USP 71 sterility testing at least 24 hours after production.

19. A sterile formulation as defined in claim 12 that is diluted in a sterile solution, and intended for administration topically to the respiratory, oropharyngeal, and/or nasopharyngeal tract, wherein the sterile formulation includes at least one additional therapeutic substance and does not require a compressed air stream for an individual to use.

20. A sterile formulation as defined in claim 12 that is diluted in a sterile solution, wherein the sterile formulation does not require a compressed air stream for an individual's use and includes at least one additional therapeutic substance.
